(19) United States
(12) Patent Application Publication
(10) Pub. No.: US 2004/0230230 A1
(43) Pub. Date: Nov. 18, 2004

(54) Methods and systems involving subcutaneous electrode positioning relative to a heart

(76) Inventors: Curtis Charles Lindstrom, Roseville, MN (US); Adam W. Cates, Minneapolis, MN (US); Darrell Orvin Wagner, Isanti, MN (US)

Correspondence Address: Crawford Maunu PLLC, Suite 390, 1270 Northland Drive, St. Paul, MN, 55120, US

(21) Appl. No.: 10/465,520

(22) Filed: Jun. 19, 2003

Related U.S. Application Data
(60) Provisional application No. 60/462,272, filed on Apr. 11, 2003.

(51) Int. Cl. A61N 1/362

(52) U.S. Cl. 607/4

(57) ABSTRACT

An approach for implementing a subcutaneous medical electrode system involves positioning a number of electrode subsystems in relation to a heart so that a majority of ventricular tissue is included within a volume defined between the electrode subsystems. One of the electrode subsystems so positioned may include a can electrode located on a housing enclosing a medical device. The medical device may be configured to provide therapeutic, diagnostic, or monitoring functions, including, for example, cardiac arrhythmia therapy.

### RELATED APPLICATIONS

This application claims the benefit of Provisional Patent Application Ser. No. 60/462,272, filed on Apr. 11, 2003, to which priority is claimed pursuant to 35 U.S.C. §119(e) and which is hereby incorporated herein by reference.

### FIELD OF THE INVENTION

The present invention relates generally to implantable medical devices and, more particularly, to subcutaneous electrode placement.

### BACKGROUND OF THE INVENTION

The healthy heart produces regular, synchronized contractions. Rhythmic contractions of the heart are normally controlled by the sinoatrial (SA) node, which are specialized cells located in the upper right atrium. The SA node is the normal pacemaker of the heart, typically initiating 60-100 heart beats per minute. When the SA node is pacing the heart normally, the heart is said to be in normal sinus rhythm.

If the heart's electrical activity becomes uncoordinated or irregular, the heart is denoted to be arrhythmic. Cardiac arrhythmia impairs cardiac efficiency and can be a potential life threatening event. Cardiac arrythmias have a number of etiological sources, including tissue damage due to myocardial infarction, infection, or degradation of the heart's ability to generate or synchronize the electrical impulses that coordinate contractions.

Bradycardia occurs when the heart rhythm is too slow. This condition may be caused, for example, by impaired function of the SA node, denoted sick sinus syndrome, or by delayed propagation or blockage of the electrical impulse between the atria and ventricles. Bradycardia produces a heart rate that is too slow to maintain adequate circulation.

When the heart rate is too rapid, the condition is denoted tachycardia. Tachycardia may have its origin in either the atria or the ventricles. Tachycardias occurring in the atria of the heart, for example, include atrial fibrillation and atrial flutter. Both conditions are characterized by rapid contractions of the atria. Besides being hemodynamically inefficient, the rapid contractions of the atria can also adversely affect the ventricular rate.

Ventricular tachycardia occurs, for example, when electrical activity arises in the ventricular myocardium at a rate more rapid than the normal sinus rhythm. Ventricular tachycardia can quickly degenerate into ventricular fibrillation. Ventricular fibrillation is a condition denoted by extremely rapid, uncoordinated electrical activity within the ventricular tissue. The rapid and erratic excitation of the ventricular tissue prevents synchronized contractions and impairs the heart's ability to effectively pump blood to the body, which is a fatal condition unless the heart is returned to sinus rhythm within a few minutes.

Implantable cardiac rhythm management systems have been used as an effective treatment for patients with serious arrhythmias. These systems typically include one or more leads and circuitry to sense signals from one or more interior and/or exterior surfaces of the heart. Such systems also include circuitry for generating electrical pulses which are applied to cardiac tissue at one or more interior and/or exterior surfaces of the heart. For example, leads extending into the patient's heart are connected to electrodes that contact the myocardium for sensing the heart's electrical signals and for delivering pulses to the heart in accordance with various therapies for treating the arrythmias described above.

Implantable cardioverter/defibrillators (ICDs) have been used as an effective treatment for patients with serious cardiac arrhythmias. For example, a typical ICD includes one or more endocardial leads to which at least one defibrillation electrode is connected. Such ICDs are capable of delivering high energy shocks to the heart, interrupting the ventricular tachyarrythmia or ventricular fibrillation, and allowing the heart to resume normal sinus rhythm. ICDs may also include pacing functionality.

Although ICDs are very effective at preventing Sudden Cardiac Death (SCD), most people at risk of SCD are not provided with implantable defibrillators. The primary reasons for this unfortunate reality include the limited number of physicians qualified to perform transvenous lead/electrode implantation, a limited number of surgical facilities adequately equipped to accommodate such cardiac procedures, and a limited number of the at-risk patient population that can safely undergo the required endocardial or epicardial lead/electrode implant procedure.

For reasons stated above, and for other reasons which will become apparent to those skilled in the art upon reading the present specification, there is a need for systems and methods that provide for sensing cardiac activity without the need for endocardial or epicardial leads/electrodes. There is a further need for systems and methods that provide for delivering cardiac stimulation therapy without the need for endocardial or epicardial leads/electrodes. The present invention fulfills these and other needs, and addresses deficiencies in known systems and techniques.

### SUMMARY OF THE INVENTION

The present invention is directed to cardiac stimulation methods and systems that, in general, provide transthoracic defibrillation therapies, transthoracic pacing therapies, or a combination of transthoracic defibrillation and pacing therapies. Embodiments of the present invention include those directed to subcutaneous cardiac stimulation methods and systems that detect and treat cardiac arrhythmia.

According to one embodiment of the invention, a medical system includes a housing having a medical device disposed within the housing. The medical device is coupled to subcutaneous electrode subsystems positioned relative to a heart so that a majority of ventricular tissue is included within a volume defined between the electrode subsystems.

In another embodiment of the invention, a medical device is disposed within a housing including a can electrode. The medical device is coupled to the can electrode and to a subcutaneous electrode subsystem. The can electrode and the electrode subsystem are positioned relative to a heart so that a majority of ventricular tissue is included within a volume defined between the can electrode and the electrode subsystem.

Yet another embodiment of the invention involves a medical system including a housing having a medical device disposed within and first and second subcutaneous electrode subsystems coupled to the medical device. The first and the second subcutaneous electrode subsystems are positioned relative to a heart so that a majority of ventricular tissue is included within a volume defined between the first and the second electrode subsystems.

In a further embodiment of the invention, an electrode system includes a first subcutaneous electrode subsystem and a second subcutaneous electrode subsystem. The first and the second electrode subsystems are positioned so that a majority of ventricular tissue is included within a volume defined between the first and the second electrode subsystems.

In yet another embodiment of the invention, a method involves coupling subcutaneous electrode subsystems to a medical device disposed within a housing and positioning the electrode subsystems in relation to a heart so that a majority of ventricular tissue is included within a volume region between the electrode subsystems.

In accordance with a further embodiment of the invention, a medical device involves means for sensing physiological conditions and means for detecting cardiac arrhythmia based on the sensed physiological conditions. The medical device also includes means for electrically stimulating a heart to mitigate the cardiac arrhythmia. The means for electrically stimulating the heart are positioned subcutaneously in relation to the heart so that a majority of ventricular tissue is included within a volume defined between the means for electrically stimulating the heart.

The above summary of the present invention is not intended to describe each embodiment or every implementation of the present invention. Advantages and attainments, together with a more complete understanding of the invention, will become apparent and appreciated by referring to the following detailed description and claims taken in conjunction with the accompanying drawings.

### BRIEF DESCRIPTION OF THE DRAWINGS

FIGS. 1A and 1B are views of a transthoracic cardiac sensing and/or stimulation device as implanted in a patient in accordance with an embodiment of the present invention;

FIG. 1C is a block diagram showing various components of a transthoracic cardiac sensing and/or stimulation device in accordance with an embodiment of the present invention;

FIG. 1D is a block diagram illustrating various processing and detection components of a transthoracic cardiac sensing and/or stimulation device in accordance with an embodiment of the present invention;

FIG. 1E is a block diagram showing various sensors, devices, and circuitry of a transthoracic cardiac sensing and/or stimulation device in accordance with an embodiment of the present invention;

FIGS. 2A-C are diagrams illustrating various components of a transthoracic cardiac sensing and/or stimulation device positioned in accordance with embodiments of the invention;

FIGS. 3A-C are diagrams illustrating electrode subsystem placement relative to a heart in accordance with embodiments of the invention; and

FIGS. 4A-F are diagrams illustrating various examples of sensing and stimulation electrode arrangements that may be implemented in electrode subsystems configured in accordance with embodiments of the invention.

While the invention is amenable to various modifications and alternative forms, specifics thereof have been shown by way of example in the drawings and will be described in detail below. It is to be understood, however, that the intention is not to limit the invention to the particular embodiments described. On the contrary, the invention is intended to cover all modifications, equivalents, and alternatives falling within the scope of the invention as defined by the appended claims.

### DETAILED DESCRIPTION OF VARIOUS EMBODIMENTS

In the following description of the illustrated embodiments, references are made to the accompanying drawings which form a part hereof, and in which is shown by way of illustration, various embodiments in which the invention may be practiced. It is to be understood that other embodiments may be utilized, and structural and functional changes may be made without departing from the scope of the present invention.

An implanted device can include one or more of the features, structures, methods, or combinations thereof described hereinbelow. For example, a cardiac monitor or a cardiac stimulator can be implemented to include one or more of the advantageous features and/or processes described below. It is intended that such a monitor, stimulator, or other implanted or partially implanted device need not include all of the features described herein, but can be implemented to include selected features that provide for unique structures and/or functionality. Such a device may be implemented to provide a variety of therapeutic or diagnostic functions.

One such device, termed an implantable transthoracic cardiac sensing and/or stimulation (ITCS) device, is described herein to include various advantageous features and/or processes. It is understood that the description of features and processes within the context of an ITCS device is provided for non-limiting illustrative purposes only, and that such features and process can be implemented in other types of devices, including implantable and non-implantable devices. For example, various features and processes described herein can be implemented in cardiac monitors, diagnostic devices, pacemakers, cardioverters/defibrillators, resynchronizers, and the like, including those devices disclosed in the various patents incorporated herein by reference. It is further understood that features and processes described herein can be implemented in devices that may employ one or more of transvenous, endocardial, epicardial, subcutaneous or surface electrodes, or devices that may employ combinations of these electrodes.

In general terms, an implantable transthoracic cardiac sensing and/or stimulation (ITCS) device can be implanted under the skin in the chest region of a patient. The ITCS device may, for example, be implanted subcutaneously such that all or selected elements of the device are positioned on the patient's front, back, side, or other body locations suitable for sensing cardiac activity and delivering cardiac stimulation therapy. It is understood that elements of the ITCS device may be located at several different body locations, such as in the chest, abdominal, or subclavian region with electrode elements respectively positioned at different regions near, around, in, or on the heart.

In one configuration, the primary housing (e.g., the active or non-active can) of the ITCS device, for example, can be configured for positioning outside of the rib cage at an intercostal or subcostal location, within the abdomen, or in the upper chest region (e.g., subclavian location, such as above the third rib). In one implementation, one or more electrodes can be located on the primary housing and/or at other locations about, but not in direct contact with the heart, great vessel or coronary vasculature. In another implementation, one or more electrodes can be located in direct contact with the heart, great vessel or coronary vasculature, such as via one or more leads. In another implementation, for example, one or more subcutaneous electrode subsystems or electrode arrays can be used to sense cardiac activity and deliver cardiac stimulation energy in an ITCS device configuration employing an active can or a configuration employing a non-active can. Electrodes can be situated at anterior and/or posterior locations relative to the heart.

Certain configurations illustrated herein are generally described as capable of implementing various functions traditionally performed by an implantable cardioverter/defibrillator (ICD), and may operate in numerous cardioversion/defibrillation modes as are known in the art. Exemplary ICD circuitry, structures and functionality, aspects of which can be incorporated in an ITCS device of a type contemplated herein, are disclosed in commonly owned U.S. Pat. Nos. 5,133,353; 5,179,945; 5,314,459; 5,318,597; 5,620,466; and 5,662,688, which are hereby incorporated herein by reference in their respective entireties.

In particular configurations, systems and methods can perform functions traditionally performed by pacemakers, such as providing various pacing therapies as are known in the art, in addition to cardioversion/defibrillation therapies. Exemplary pacemaker circuitry, structures and functionality, aspects of which can be incorporated in an ITCS device of a type contemplated herein, are disclosed in commonly owned U.S. Pat. Nos. 4,562,841; 5,284,136; 5,376,106; 5,036,849; 5,540,727; 5,836,987; 6,044,298; and 6,055,454, which are hereby incorporated herein by reference in their respective entireties. It is understood that ITCS device configurations can provide for non-physiologic pacing support in addition to, or to the exclusion of, bradycardia and/or anti-tachycardia pacing therapies.

An ITCS device can implement functionality traditionally provided by cardiac diagnostic devices or cardiac monitors as are known in the art, alternatively or additionally to providing cardioversion/defibrillation therapies. Exemplary cardiac monitoring circuitry, structures and functionality, aspects of which can be incorporated in an ITCS device of a type contemplated herein, are disclosed in commonly owned U.S. Pat. Nos. 5,313,953; 5,388,578; and 5,411,031, which are hereby incorporated herein by reference in their respective entireties.

An ITCS device may implement various anti-tachyarrhythmia therapies, such as tiered therapies, which may involve performing rate-based, pattern and rate-based, and/or morphological tachyarrhythmia discrimination analyses. Subcutaneous, cutaneous, and/or external sensors can be employed to acquire physiologic and non-physiologic information for purposes of enhancing tachyarrhythmia detection and termination. It is understood that configurations, features, and combination of features described in the instant disclosure can be implemented in a wide range of implantable medical devices, and that such embodiments and features are not limited to the particular devices described herein.

An ITCS device may be used to implement various diagnostic functions, which may involve performing rate-based, pattern and rate-based, and/or morphological tachyarrhythmia discrimination analyses. Subcutaneous, cutaneous, and/or external sensors can be employed to acquire physiologic and non-physiologic information for purposes of enhancing tachyarrhythmia detection and termination. It is understood that configurations, features, and combination of features described in the instant disclosure can be implemented in a wide range of implantable medical devices, and that such embodiments and features are not limited to the particular devices described herein.

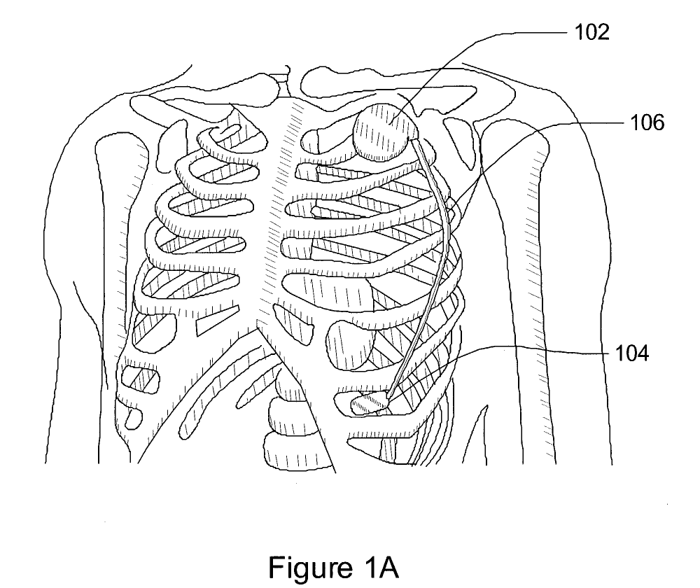

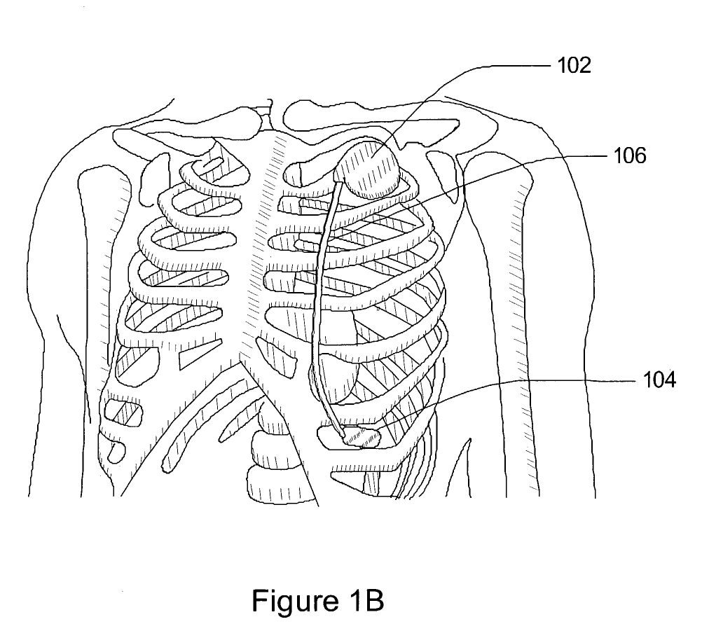

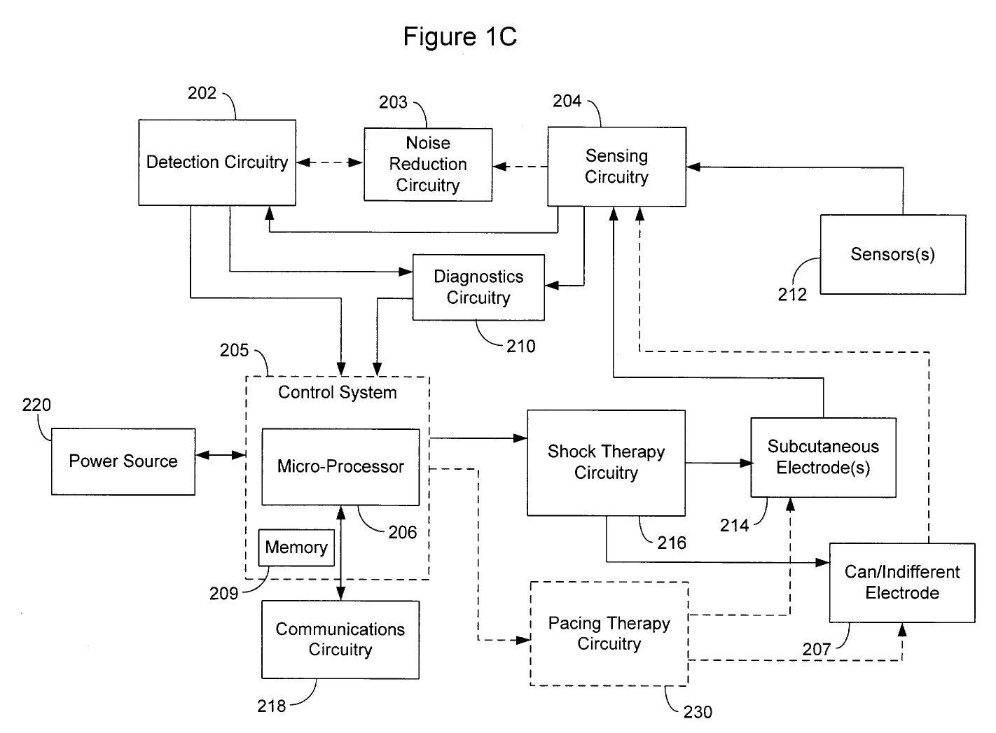

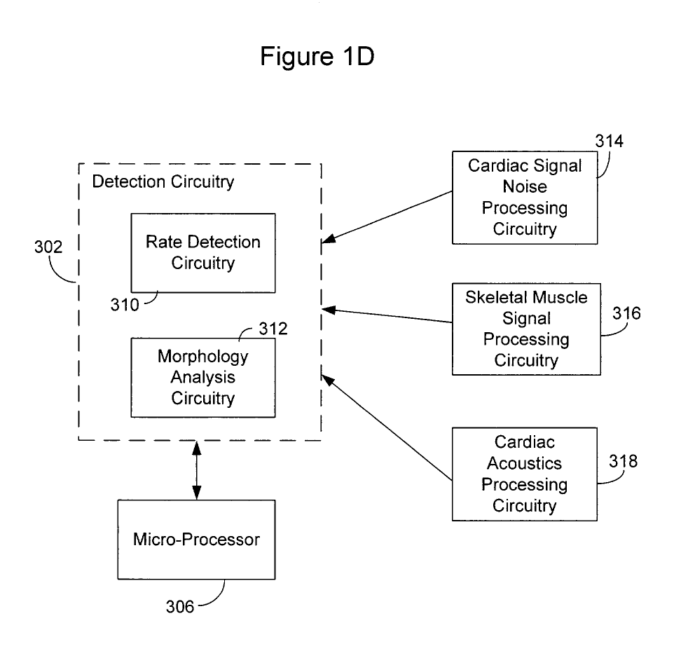

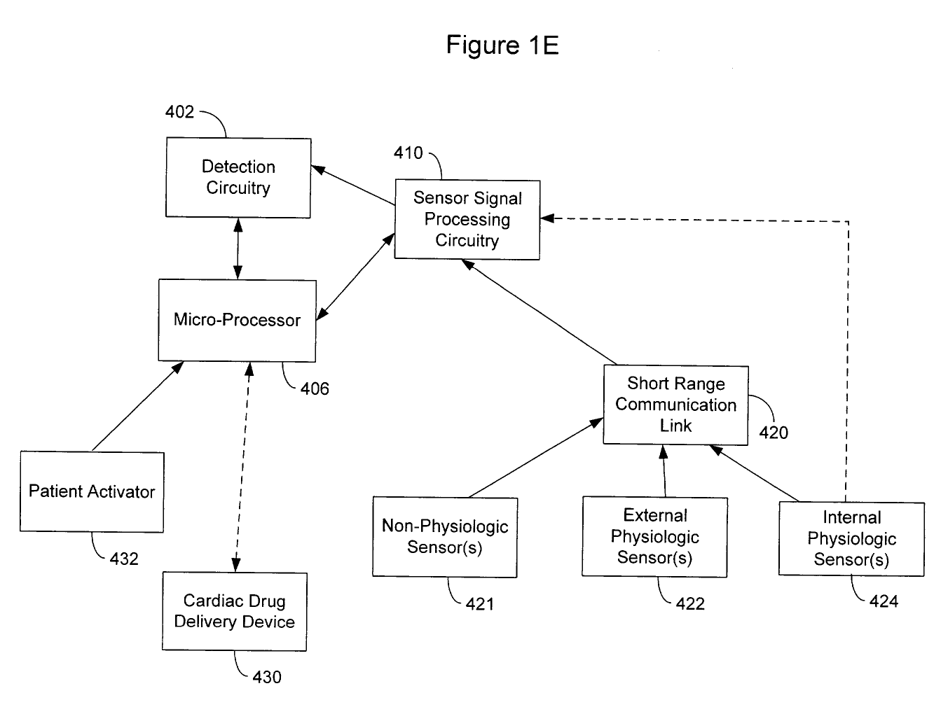

Referring now to FIGS. 1A and 1B of the drawings, there is shown a configuration of a transthoracic cardiac sensing and/or stimulation (ITCS) device implanted in the chest region of a patient at different locations. In the particular configuration shown in FIGS. 1A and 1B, the ITCS device includes a housing 102 within which various cardiac sensing, detection, processing, and energy delivery circuitry can be housed. It is understood that the components and functionality depicted in the figures and described herein can be implemented in hardware, software, or a combination of hardware and software. It is further understood that the components and functionality depicted as separate or discrete blocks/elements in the figures can be implemented in combination with other components and functionality, and that the depiction of such components and functionality in individual or integral form is for purposes of clarity of explanation, and not of limitation.

Communications circuitry is disposed within the housing 102 for facilitating communication between the ITCS device and an external communication device, such as a portable or bed-side communication station, patient-carried/worn communication station, or external programmer, for example. The communications circuitry can also facilitate unidirectional or bidirectional communication with one or more external, cutaneous, or subcutaneous physiologic or non-physiologic sensors. The housing 102 is typically configured to include one or more electrodes (e.g., can electrode and/or indifferent electrode). Although the housing 102 is typically configured as an active can, it is appreciated that a non-active can configuration may be implemented, in which case at least two electrodes spaced apart from the housing 102 are employed.

In the configuration shown in FIGS. 1A and 1B, a subcutaneous electrode 104 can be positioned under the skin in the chest region and situated distal from the housing 102. The subcutaneous and, if applicable, housing electrode(s) can be positioned about the heart at various locations and orientations, such as at various anterior and/or posterior locations relative to the heart. The subcutaneous electrode 104 is electrically coupled to circuitry within the housing 102 via a lead assembly 106. One or more conductors (e.g., coils or cables) are provided within the lead assembly 106 and electrically couple the subcutaneous electrode 104 with circuitry in the housing 102. One or more sense, sense/pace or defibrillation electrodes can be situated on the elongated structure of the electrode support, the housing 102, and/or the distal electrode assembly (shown as subcutaneous electrode 104 in the configuration shown in FIGS. 1A and 1B).

In one configuration, the lead assembly 106 is generally flexible and has a construction similar to conventional implantable, medical electrical leads (e.g., defibrillation leads or combined defibrillation/pacing leads). In another configuration, the lead assembly 106 is constructed to be somewhat flexible, yet has an elastic, spring, or mechanical memory that retains a desired configuration after being shaped or manipulated by a clinician. For example, the lead assembly 106 can incorporate a gooseneck or braid system that can be distorted under manual force to take on a desired shape. In this manner, the lead assembly 106 can be shape-fit to accommodate the unique anatomical configuration of a given patient, and generally retains a customized shape after implantation. Shaping of the lead assembly 106 according to this configuration can occur prior to, and during, ITCS device implantation.

In accordance with a further configuration, the lead assembly 106 includes a rigid electrode support assembly, such as a rigid elongated structure that positionally stabilizes the subcutaneous electrode 104 with respect to the housing 102. In this configuration, the rigidity of the elongated structure maintains a desired spacing between the subcutaneous electrode 104 and the housing 102, and a desired orientation of the subcutaneous electrode 104/housing 102 relative to the patient's heart. The elongated structure can be formed from a structural plastic, composite or metallic material, and comprises, or is covered by, a biocompatible material. Appropriate electrical isolation between the housing 102 and subcutaneous electrode 104 is provided in cases where the elongated structure is formed from an electrically conductive material, such as metal.

In one configuration, the rigid electrode support assembly and the housing 102 define a unitary structure (i.e., a single housing/unit). The electronic components and electrode conductors/connectors are disposed within or on the unitary ITCS device housing/electrode support assembly. At least two electrodes are supported on the unitary structure near opposing ends of the housing/electrode support assembly. The unitary structure can have an arcuate or angled shape, for example.

According to another configuration, the rigid electrode support assembly defines a physically separable unit relative to the housing 102. The rigid electrode support assembly includes mechanical and electrical couplings that facilitate mating engagement with corresponding mechanical and electrical couplings of the housing 102. For example, a header block arrangement can be configured to include both electrical and mechanical couplings that provide for mechanical and electrical connections between the rigid electrode support assembly and housing 102. The header block arrangement can be provided on the housing 102 or the rigid electrode support assembly. Alternatively, a mechanical/electrical coupler can be used to establish mechanical and electrical connections between the rigid electrode support assembly and housing 102. In such a configuration, a variety of different electrode support assemblies of varying shapes, sizes, and electrode configurations can be made available for physically and electrically connecting to a standard ITCS device housing 102.

It is noted that the electrodes and the lead assembly 106 can be configured to assume a variety of shapes. For example, the lead assembly 106 can have a wedge, chevron, flattened oval, or a ribbon shape, and the subcutaneous electrode 104 can comprise a number of spaced electrodes, such as an array or band of electrodes. Moreover, two or more subcutaneous electrodes 104 can be mounted to multiple electrode support assemblies 106 to achieve a desired spaced relationship amongst subcutaneous electrodes 104.

An ITCS device can incorporate circuitry, structures and functionality of the subcutaneous implantable medical devices disclosed in commonly owned U.S. Pat. Nos. 5,203,348; 5,230,337; 5,360,442; 5,366,496; 5,397,342; 5,391,200; 5,545,202; 5,603,732; and 5,916,243, which are hereby incorporated herein by reference in their respective entireties.

Depending on the configuration of a particular ITCS device, a delivery system can advantageously be used to facilitate proper placement and orientation of the ITCS device housing and subcutaneous electrode(s). According to one configuration of such a delivery system, a long metal rod similar to conventional trocars can be used to perform small diameter blunt tissue dissection of the subdermal layers. This tool may be pre-formed straight or curved to facilitate placement of the subcutaneous electrode, or it may be flexible enough to allow the physician to shape it appropriately for a given patient. An exemplary delivery tool, aspects of which can be incorporated into an ITCS device delivery tool, is disclosed in commonly owned U.S. Pat. No. 5,300,106, which is hereby incorporated herein by reference in its entirety.

FIG. 1C is a block diagram depicting various components of an ITCS device in accordance with one configuration. According to this configuration, the ITCS device incorporates a processor-based control system 205 which includes a micro-processor 206 coupled to appropriate memory (volatile and non-volatile) 209, it being understood that any logic-based control architecture can be used. The control system 205 is coupled to circuitry and components to sense, detect, and analyze electrical signals produced by the heart and deliver electrical stimulation energy to the heart under predetermined conditions to treat cardiac arrhythmias. In certain configurations, the control system 205 and associated components also provide pacing therapy to the heart. The electrical energy delivered by the ITCS device may be in the form of low energy pacing pulses or high energy pulses for cardioversion or defibrillation.

Cardiac signals are sensed using the subcutaneous electrode(s) 214 and the can or indifferent electrode 207 provided on the ITCS device housing. Cardiac signals can also be sensed using only the subcutaneous electrodes 214, such as in a non-active can configuration. As such, unipolar, bipolar, or combined unipolar/bipolar electrode configurations may be employed. The sensed cardiac signals are received by sensing circuitry 204, which includes sense amplification circuitry and may also include filtering circuitry and an analog-to-digital (A/D) converter. The sensed cardiac signals processed by the sensing circuitry 204 may be received by noise reduction circuitry 203, which can further reduce noise before signals are sent to the detection circuitry 202. Noise reduction circuitry 203 may also be incorporated after detection circuitry 202 in cases where high power or computationally intensive noise reduction algorithms are required.

In the illustrative configuration shown in FIG. 1C, the detection circuitry 202 is coupled to, or otherwise incorporates, noise reduction circuitry 203. The noise reduction circuitry 203 operates to improve the signal-to-noise ratio of sensed cardiac signals by removing noise content of the sensed cardiac signals introduced from various sources. Typical types of transthoracic cardiac signal noise includes electrical noise and noise produced from skeletal muscles, for example. A number of methodologies for improving the signal-to-noise ratio of sensed cardiac signals in the presence of skeletal muscular induced noise, including signal separation techniques, are described hereinbelow.

According to another aspect, skeletal muscular noise can be used as a useful artifact signal for a variety of purposes. In one approach, the detection circuitry 202 and noise reduction circuitry 203 cooperate to detect skeletal muscular noise, and the detected skeletal muscular noise can be used to determine the activity level of the patient. The activity level information derived from the detected skeletal muscular noise can be used for a number of purposes, such as minimizing the delivery of inappropriate cardioversion and defibrillation therapy, as is discussed in greater detail hereinbelow.

Detection circuitry 202 typically includes a signal processor that coordinates analysis of the sensed cardiac signals and/or other sensor inputs to detect cardiac arrhythmias, such as, in particular, tachyarrhythmia. Rate based and/or morphological discrimination algorithms can be implemented by the signal processor of the detection circuitry 202 to detect and verify the presence and severity of an arrhythmic episode. Exemplary arrhythmia detection and discrimination circuitry, structures, and techniques, aspects of which can be implemented by an ITCS device of a type contemplated herein, are disclosed in commonly owned U.S. Pat. Nos. 5,301,677 and 6,438,410, which are hereby incorporated herein by reference in their respective entireties. Arrhythmia detection methodologies particularly well suited for implementation in subcutaneous cardiac stimulation systems are described hereinbelow.

The detection circuitry 202 communicates cardiac signal information to the control system 205. Memory circuitry 209 of the control system 205 contains parameters for operating in various sensing, defibrillation, and pacing modes, and stores data indicative of cardiac signals received by the detection circuitry 202. The memory circuitry 209 can also be configured to store historical ECG and therapy data, which may be used for various purposes and transmitted to an external receiving device as needed or desired.

In certain configurations, the ITCS device can include diagnostics circuitry 210. The diagnostics circuitry 210 typically receives input signals from the detection circuitry 202 and the sensing circuitry 204. The diagnostics circuitry 210 provides diagnostics data to the control system 205, it being understood that the control system 205 can incorporate all or part of the diagnostics circuitry 210 or its functionality. The control system 205 may store and use information provided by the diagnostics circuitry 210 for a variety of diagnostics purposes. This diagnostic information may be stored, for example, subsequent to a triggering event or at predetermined intervals, and may include system diagnostics, such as power source status, therapy delivery history, and/or patient diagnostics. The diagnostic information may take the form of electrical signals or other sensor data acquired immediately prior to therapy delivery.

According to a configuration that provides cardioversion and defibrillation therapies, the control system 205 processes cardiac signal data received from the detection circuitry 202 and initiates appropriate tachyarrhythmia therapies to terminate cardiac arrhythmic episodes and return the heart to normal sinus rhythm. The control system 205 is coupled to shock therapy circuitry 216. The shock therapy circuitry 216 is coupled to the subcutaneous electrode(s) 214 and the can or indifferent electrode 207 of the ITCS device housing. Upon command, the shock therapy circuitry 216 delivers cardioversion and defibrillation stimulation energy to the heart in accordance with a selected cardioversion or defibrillation therapy. In a less sophisticated configuration, the shock therapy circuitry 216 is controlled to deliver defibrillation therapies, in contrast to a configuration that provides for delivery of both cardioversion and defibrillation therapies. Exemplary ICD high energy delivery circuitry, structures and functionality, aspects of which can be incorporated in an ITCS device of a type contemplated herein, are disclosed in commonly owned U.S. Pat. Nos. 5,372,606; 5,411,525; 5,468,254; and 5,634,938, which are hereby incorporated herein by reference in their respective entireties.

In accordance with another configuration, an ITCS device can incorporate a cardiac pacing capability in addition to cardioversion and/or defibrillation capabilities. As is shown in dotted lines in FIG. 1C, the ITCS device can include pacing therapy circuitry 230 which is coupled to the control system 205 and the subcutaneous and can/indifferent electrodes 214, 207. Upon command, the pacing therapy circuitry delivers pacing pulses to the heart in accordance with a selected pacing therapy. Control signals, developed in accordance with a pacing regimen by pacemaker circuitry within the control system 205, are initiated and transmitted to the pacing therapy circuitry 230 where pacing pulses are generated. A pacing regimen may be modified by the control system 205.

A number of cardiac pacing therapies are described herein which are particularly useful in a transthoracic cardiac stimulation device. Such cardiac pacing therapies can be delivered via the pacing therapy circuitry 230 as shown in FIG. 1C. Alternatively, cardiac pacing therapies can be delivered via the shock therapy circuitry 216, which effectively obviates the need for separate pacemaker circuitry.

The ITCS device shown in FIG. 1C can be configured to receive signals from one or more physiologic and/or non-physiologic sensors. Depending on the type of sensor employed, signals generated by the sensors can be communicated to transducer circuitry coupled directly to the detection circuitry or indirectly via the sensing circuitry. It is noted that certain sensors can transmit sense data to the control system 205 without processing by the detection circuitry 202.

Communications circuitry 218 is coupled to the micro-processor 206 of the control system 205. The communications circuitry 218 allows the ITCS device to communicate with one or more receiving devices or systems situated external to the ITCS device. By way of example, the ITCS device can communicate with a patient-worn, portable or bed-side communication system via the communications circuitry 218. In one configuration, one or more physiologic or non-physiologic sensors (subcutaneous, cutaneous, or external of patient) can be equipped with a short-range wireless communication interface, such as an interface conforming to a known communications standard, such as Bluetooth or IEEE 802 standards. Data acquired by such sensors can be communicated to the ITCS device via the communications circuitry 218. It is noted that physiologic or non-physiologic sensors equipped with wireless transmitters or transceivers can communicate with a receiving system external of the patient.

The communications circuitry 218 can allow the ITCS device to communicate with an external programmer. In one configuration, the communications circuitry 218 and the programmer unit (not shown) use a wire loop antenna and a radio frequency telemetric link, as is known in the art, to receive and transmit signals and data between the programmer unit and communications circuitry 218. In this manner, programming commands and data are transferred between the ITCS device and the programmer unit during and after implant. Using a programmer, a physician is able to set or modify various parameters used by the ITCS device. For example, a physician can set or modify parameters affecting sensing, detection, pacing, and defibrillation functions of the ITCS device, including pacing and cardioversion/defibrillation therapy modes.

Typically, the ITCS device is encased and hermetically sealed in a housing suitable for implanting in a human body as is known in the art. Power to the ITCS device is supplied by an electrochemical power source 220 housed within the ITCS device. In one configuration, the power source 220 includes a rechargeable battery. According to this configuration, charging circuitry is coupled to the power source 220 to facilitate repeated non-invasive charging of the power source 220. The communications circuitry 218, or separate receiver circuitry, is configured to receive RF energy transmitted by an external RF energy transmitter. The ITCS device may, in addition to a rechargeable power source, include a non-rechargeable battery. It is understood that a rechargeable power source need not be used, in which case a long-life non-rechargeable battery is employed.

FIG. 1D illustrates a configuration of detection circuitry 302 of an ITCS device which includes one or both of rate detection circuitry 310 and morphological analysis circuitry 312. Detection and verification of arrhythmias can be accomplished using rate-based discrimination algorithms as known in the art implemented by the rate detection circuitry 310. Arrhythmic episodes can also be detected and verified by morphology-based analysis of sensed cardiac signals as is known in the art. Tiered or parallel arrhythmia discrimination algorithms can also be implemented using both rate-based and morphologic-based approaches. Further, a rate and pattern-based arrhythmia detection and discrimination approach may be employed to detect and/or verify arrhythmic episodes, such as the approach disclosed in U.S. Pat. Nos. 6,487,443; 6,259,947; 6,141,581; 5,855,593; and 5,545,186, which are hereby incorporated herein by reference in their respective entireties.

The detection circuitry 302, which is coupled to a micro-processor 306, can be configured to incorporate, or communicate with, specialized circuitry for processing sensed cardiac signals in manners particularly useful in a transthoracic cardiac stimulation device. As is shown by way of example in FIG. 1D, the detection circuitry 302 can receive information from multiple physiologic and non-physiologic sensors. As illustrated, transthoracic acoustics can be monitored using an appropriate acoustic sensor. Heart sounds, for example, can be detected and processed by cardiac acoustic processing circuitry 318 for a variety of purposes. The acoustics data is transmitted to the detection circuitry 302, via a hardwire or wireless link, and used to enhance cardiac signal detection. For example, acoustics can be used to discriminate normal cardiac sinus rhythm with electrical noise from potentially lethal arrhythmias, such as ventricular tachycardia or ventricular fibrillation.

The detection circuitry 302 can also receive information from one or more sensors that monitor skeletal muscle activity. In addition to cardiac activity signals, skeletal muscle signals are readily detected by transthoracic electrodes. Such skeletal muscle signals can be used to determine the activity level of the patient. In the context of cardiac signal detection, such skeletal muscle signals are considered artifacts of the cardiac activity signal, which can be viewed as noise. Processing circuitry 316 receives signals from one or more skeletal muscle sensors, and transmits processed skeletal muscle signal data to the detection circuitry 302. This data can be used to discriminate normal cardiac sinus rhythm with skeletal muscle noise from cardiac arrhythmias.

As was previously discussed, the detection circuitry 302 is coupled to, or otherwise incorporates, noise processing circuitry 314. The noise processing circuitry 314 processes sensed cardiac signals to improve the signal-to-noise ratio of sensed cardiac signals by removing noise content of the sensed cardiac signals.

Turning now to FIG. 1E, there is illustrated a block diagram of various components of an ITCS device in accordance with one configuration. FIG. 1E shows a number of components that are associated with detection of various physiologic and non-physiologic parameters. As shown, the ITCS device includes a micro-processor 406, which is typically incorporated in a control system for the ITCS device, coupled to detection circuitry 402. Sensor signal processing circuitry 410 can receive sensor data from a number of different sensors.

For example, an ITCS device can cooperate with, or otherwise incorporate, various types of non-physiologic sensors 421, external/cutaneous physiologic sensors 422, and/or internal physiologic sensors 424. Such sensors can include an acoustic sensor, an impedance sensor, an oxygen saturation sensor, and a blood pressure sensor, for example. Each of these sensors 421, 422, 424 can be communicatively coupled to the sensor signal processing circuitry 410 via a short range wireless communication link 420. Certain sensors, such as an internal physiologic sensor 424, can alternatively be communicatively coupled to the sensor signal processing circuitry 410 via a wired connection (e.g., electrical or optical connection).

A cardiac drug delivery device 430 can be employed to cooperate with an ITCS device of a type contemplated herein. For example, the cardiac drug delivery device 430 can deliver one or more anti-arrhythmic agents that have been approved for the chemical treatment of tachycardia and fibrillation. A non-exhaustive, non-limiting list of such agents includes: quinidine, procainamide, disopyramide, flecaininde, propafenone, moricizine, sotalol, amiodarone, ibutilide, dofetilide or other anti-arrhythmic agents. These and other drugs can be delivered prior to, during, and after delivery of cardioversion/defibrillation therapy for purposes of enhancing patient comfort, lowering defibrillation thresholds, and/or chemically treating an arrhythmic condition.

In accordance with another configuration, the ITCS device can include a non-implanted patient actuatable activator 432 that operates in cooperation with the ITCS device. The activator 432 includes a communication unit and produces an activation signal in response to a patient sensing a perceived severe arrhythmic condition. Alternatively, or in addition, the activation signal may be produced by the non-implanted activator 432 in response to the ITCS device detecting the arrhythmic condition. The ITCS device includes communication circuitry for communicating with the non-implanted activator 432.

The activator 432 can be actuated by the patient or person attending the patient to initiate cardioversion/defibrillation therapy. Typically, the ITCS device, in response to receiving an activation signal, confirms that the patient is experiencing an actual adverse cardiac condition prior to initiating appropriate therapy. The non-implanted activator 432, in communication with the ITCS device, can also generate a patient perceivable initiating signal to indicate manual or automatic commencement of a drug delivery regimen to treat the actual adverse cardiac condition.

The activator 432 can be configured to include an inhibit button that allows the patient to override the delivery of a stimulation therapy in the event that the ITCS device indicates that a potentially serious arrhythmia has been detected, but the patient determines that the detection indication is in error. Unambiguous arrhythmic episodes detected by the ITCS device are preferably subject to therapy delivery upon detection and confirmation, notwithstanding receipt of an inhibition signal from the patient activator 432.

The components, functionality, and structural configurations depicted in FIGS. 1A-1E are intended to provide an understanding of various features and combination of features that can be incorporated in an ITCS device. It is understood that a wide variety of ITCS and other implantable cardiac monitoring/stimulation device configurations are contemplated, ranging from relatively sophisticated to relatively simple designs. As such, particular ITCS or cardiac monitoring/stimulation device configurations can include particular features as described herein, while other such device configurations can exclude particular features described herein.

In accordance with embodiments of the invention, an ITCS device can be implemented to include a subcutaneous electrode system that provides for cardiac sensing and arrhythmia therapy. According to this approach, an ITCS device may be implemented as a chronically implantable system that performs monitoring, diagnostic and/or therapeutic functions. The ITCS device may automatically detect and treat cardiac arrhythmias. In one configuration, the ITCS device includes a pulse generator and one or more electrodes that are implanted subcutaneously in the chest region of the body, such as in the anterior thoracic region of the body. The ITCS device can be used to provide atrial and ventricular therapy for bradycardia and tachycardia arrhythmias. Tachyarrhythmia therapy can include cardioversion, defibrillation and anti-tachycardia pacing (ATP), for example, to treat atrial or ventricular tachycardia or fibrillation. Bradycardia therapy can include temporary post-shock pacing for bradycardia or asystole. Methods and systems for implementing post-shock pacing for bradycardia or asystole are described in commonly owned U.S. patent application entitled “SUBCUTANEOUS CARDIAC STIMULATOR EMPLOYING POST-SHOCK TRANSTHORACIC ASYSTOLE PREVENTION PACING, Ser. No. 10/377,274, filed on Feb. 28, 2003, which is incorporated herein by reference in its entirety.

In one configuration, an ITCS device according to this approach can utilize conventional pulse generator and subcutaneous electrode implant techniques. The pulse generator device and electrodes may be chronically implanted subcutaneously. Such an ITCS can be used to automatically detect and treat arrhythmias similarly to conventional implantable systems. In another configuration, the ITCS device may comprise a unitary structure (i.e., a single housing/unit). The electronic components and electrode conductors/connectors are disposed within or on the unitary ITCS device housing/electrode support assembly.

The ITCS device contains the electronics and can be similar to a conventional implantable defibrillator. High voltage shock therapy can be delivered between two or more electrodes, one of which may be the pulse generator housing (i.e., can), placed subcutaneously in the thoracic region of the body.

Additionally or alternatively, the ITCS device may also provide lower energy electrical stimulation for bradycardia therapy. The ITCS device may provide brady pacing similarly to a conventional pacemaker. The ITCS device may provide temporary post-shock pacing for bradycardia or asystole. Sensing and/or pacing can be accomplished using sense/pace electrodes positioned on an electrode subsystem also incorporating shock electrodes, or by separate electrodes implanted subcutaneously.

The ITCS device may detect a variety of physiological signals that may be used in connection with various diagnostic, therapeutic or monitoring implementations. For example, the ITCS device may include sensors or circuitry for detecting respiratory system signals, cardiac system signals, and signals related to patient activity. In one embodiment, the ITCS device senses intrathoracic impedance, from which various respiratory parameters may be derived, including, for example, respiratory tidal volume and minute ventilation. Sensors and associated circuitry may be incorporated in connection with an ITCS device for detecting one or more body movement or body position related signals. For example, accelerometers and GPS devices may be employed to detect patient activity, patient location, body orientation, or torso position.

The ITCS device may be used within the structure of an advanced patient management (APM) system. Advanced patient management systems may allow physicians to remotely and automatically monitor cardiac and respiratory functions, as well as other patient conditions. In one example, implantable cardiac rhythm management systems, such as cardiac pacemakers, defibrillators, and resynchronization devices, may be equipped with various telecommunications and information technologies that enable real-time data collection, diagnosis, and treatment of the patient.

An ITCS device according to this approach provides an easy to implant therapeutic, diagnostic or monitoring system. The ITCS system could potentially be implanted without the need for intravenous or intrathoracic access, providing a simpler, less invasive implant procedure and minimizing lead and surgical complications. In addition, this system would have advantages for use in patients for whom transvenous lead systems cause complications. Such complications include, but are not limited to, surgical complications, infection, insufficient vessel patency, complications associated with the presence of artificial valves, and limitations in pediatric patients due to patient growth, among others. An ITCS system according to this approach is distinct from conventional approaches in that it is preferably configured to include a combination of two or more electrode subsystems that are implanted subcutaneously in the anterior thorax.

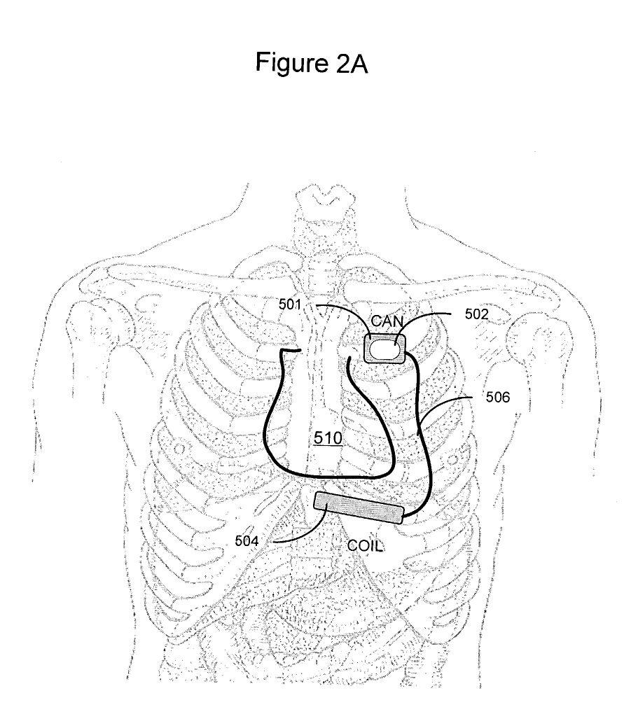

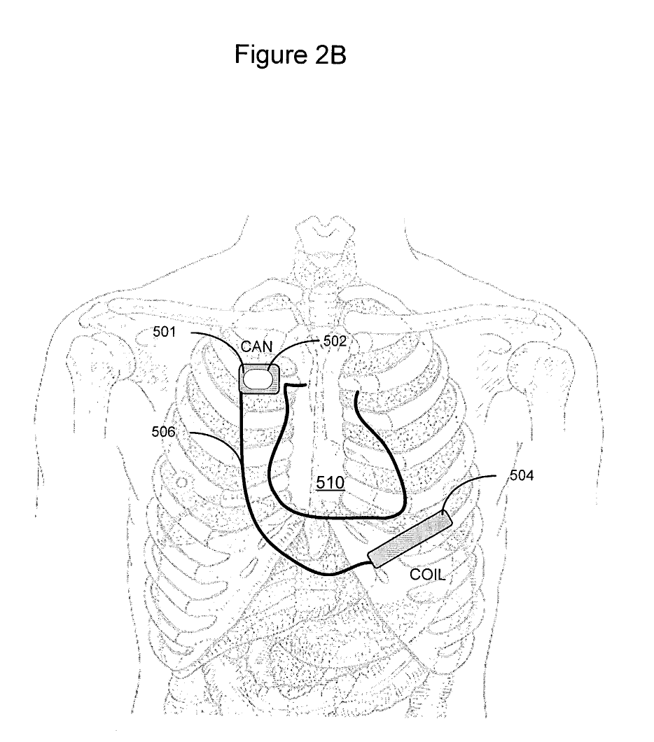

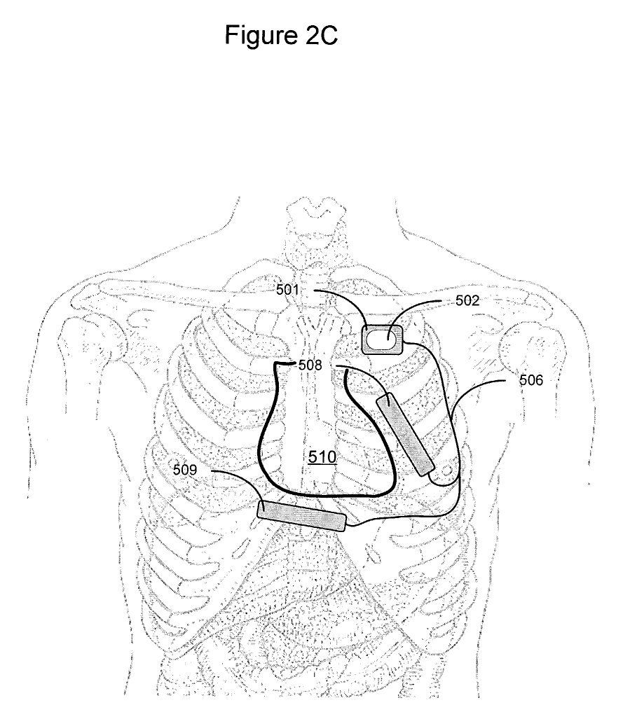

In one configuration, illustrated in FIG. 2A, electrode subsystems of the ITCS system include a first electrode subsystem, comprising a can electrode 502, and a second electrode subsystem 504 that may include at least one coil electrode, for example. The second electrode subsystem 504 may comprise a number of electrodes used for sensing and/or electrical stimulation. In various configurations, the second electrode subsystem 504 may comprise a single electrode or a combination of electrodes. The single electrode or combination of electrodes comprising the second electrode subsystem 504 may include coil electrodes, tip electrodes, ring electrodes, multi-element coils, spiral coils, spiral coils mounted on non-conductive backing, and screen patch electrodes, for example. A suitable non-conductive backing material is silicone rubber, for example.

The can electrode 502 is positioned on the housing 501 that encloses the ITCS device electronics. In one embodiment, the can electrode 502 comprises the entirety of the external surface of housing 501. In other embodiments, various portions of the housing 501 may be electrically isolated from the can electrode 502 or from tissue. For example, the active area of the can electrode 502 may comprise all or a portion of either the anterior or posterior surface of the housing 501 to direct current flow in a manner advantageous for cardiac sensing and/or stimulation.

The housing 501 may resemble that of a conventional implantable ICD, is approximately 20-100 cc in volume, with a thickness of 0.4 to 2 cm and with a surface area on each face of approximately 30 to 100 cm². As previously discussed, portions of the housing may be electrically isolated from tissue to optimally direct current flow. For example, portions of the housing 501 may be covered with a non-conductive, or otherwise electrically resistive, material to direct current flow. Suitable non-conductive material coatings include those formed from silicone rubber, polyurethane, or parylene, for example.

In addition, or alternatively, all or portions of the housing 501 may be treated to change the electrical conductivity characteristics thereof for purposes of optimally directing current flow. Various known techniques can be employed to modify the surface conductivity characteristics of the housing 501, such as by increasing or decreasing surface conductivity, to optimize current flow. Such techniques can include those that mechanically or chemically alter the surface of the housing 501 to achieve desired electrical conductivity characteristics.

FIG. 2A illustrates the housing 501 and can electrode 502 placed subcutaneously, superior to the heart 510 in the left pectoral region, which is a location commonly used for conventional pacemaker and defibrillator implants. The second electrode subsystem 504 preferably includes a coil electrode mounted on the distal end of a lead body 506, where the coil is approximately 3-15 French in diameter and 5-12 cm in length. The coil electrode may have a slight preformed curve along its length. The lead may be introduced through the lumen of a subcutaneous sheath, through a common tunneling implant technique, and the second electrode subsystem 504, e.g., comprising a coil electrode, may be placed subcutaneously, deep to any subcutaneous fat and adjacent to the underlying muscle layer.

In this configuration, the second electrode subsystem 504 is positioned approximately parallel with the inferior aspect of the right ventricle of the heart 510, just inferior to the right ventricular free wall, with one end extending just past the apex of the heart 510. For example, the tip of the electrode subsystem 504 may extend less than about 3 cm and preferably about 1-2 cm left lateral to the apex of the heart 510. The apex location may be identified by fluoroscopy or other means. This electrode arrangement may be used to include a majority of ventricular tissue within a volume defined between the housing 501 and the second electrode subsystem 504. In one configuration, a majority of the ventricular tissue is included within a volume associated with an area bounded by lines drawn between the distal and proximal ends of the second electrode subsystem 504 and the medial and lateral edges of the left pectoral can electrode 502.

In one example arrangement, the volume including a majority of ventricular tissue may be associated with a cross sectional area bounded by lines drawn between the ends of the electrode subsystems 502, 504 or between active elements of the electrode subsystems 502, 504. In one implementation, the lines drawn between active elements of the electrode subsystems 502, 504 may include a medial edge and a lateral edge of the can electrode 502, and a proximal end and a distal end of a coil electrode utilized within the second electrode subsystem 504. Arranging the electrode subsystems so that a majority of ventricular tissue is contained within a volume defined between the active elements of the electrode subsystems 502, 504 provides an efficient position for defibrillation by increasing the voltage gradient in the ventricles of the heart 510 for a given applied voltage between electrode subsystems 502, 504.

In a similar configuration, and as shown in FIG. 2B, the housing 501 comprising the can electrode 502 is placed in the right pectoral region. The second electrode subsystem 504 is positioned more laterally, to again include a majority of the ventricular tissue in a volume defined between the can electrode 502 and the second electrode subsystem 504.

In a further configuration, and as shown in FIG. 2C, the ITCS device housing 501 containing the electronics (i.e., the can) is not used as an electrode. In this case, an electrode system comprising two electrode subsystems 508, 509 coupled to the housing 501 may be implanted subcutaneously in the chest region of the body, such as in the anterior thorax. The first and the second electrode subsystems 508, 509 are placed in opposition with respect to the ventricles of the heart 510, with the majority of the ventricular tissue of the heart 510 included within a volume defined between the electrode subsystems 508, 509. As illustrated in FIG. 2C, the first electrode system 508 is positioned superior to the heart 510 relative to a superior aspect of the heart 510, e.g., parallel to the left ventricular free wall. The second electrode system 509 is located inferior to the heart 510 and positioned in relation to an inferior aspect of the heart 510, e.g., parallel to the right ventricular free wall.

In this configuration, the first and the second electrode subsystems 508. 509 may comprise any combination of electrodes used for sensing and/or electrical stimulation. In various configurations, the electrode subsystems 508, 509 may each be comprised of a single electrode or a combination of electrodes. The electrode or electrodes comprising the first and second electrode subsystems 508, 509 may include any combination of one or more coil electrodes, tip electrodes, ring electrodes, multi-element coils, spiral coils, spiral coils mounted on non-conductive backing, and screen patch electrodes, for example.

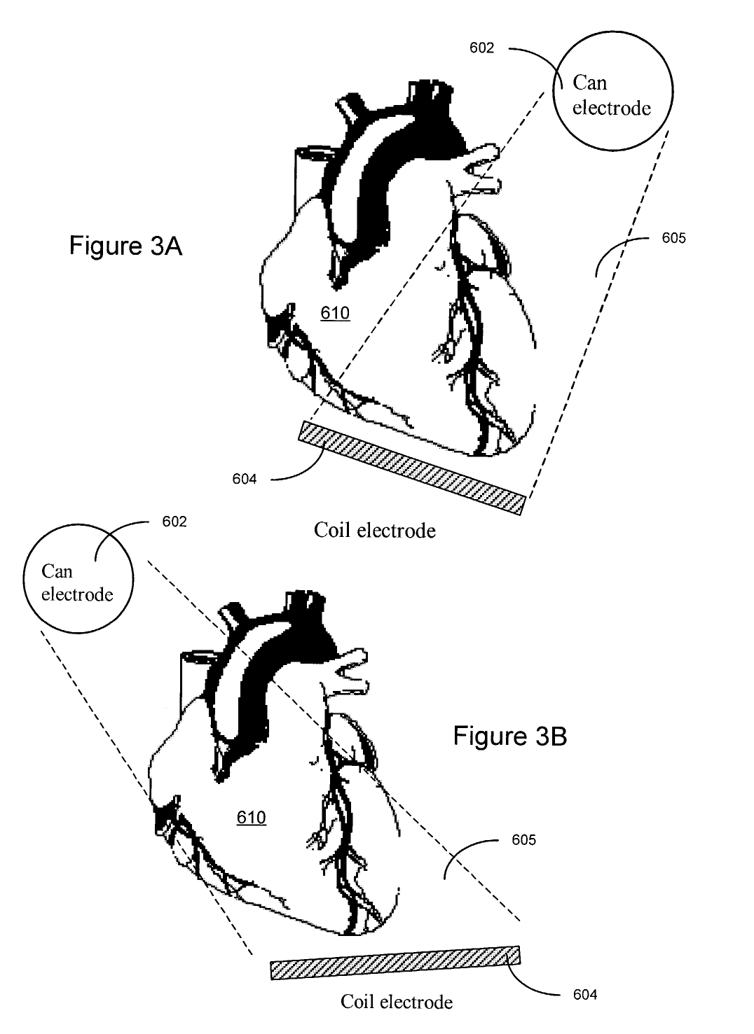

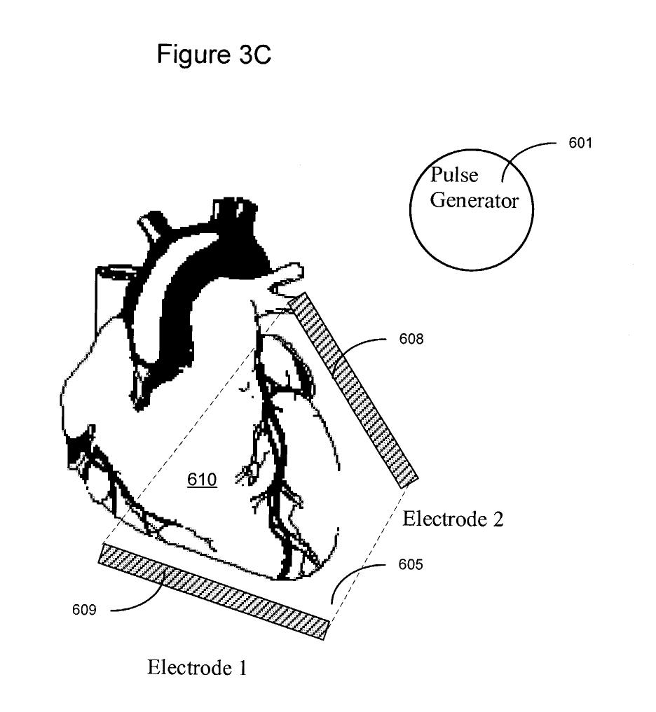

FIGS. 3A-C provide more detailed views of subcutaneous electrode subsystem placement in accordance with embodiments of the invention. FIG. 3A illustrates first and second electrode subsystems configured as a can electrode 602 and a coil electrode 604, respectively. FIG. 3A illustrates the can electrode 602 positioned superior to the heart 610 in the left pectoral region and the coil electrode 604 positioned inferior to the heart 610, parallel to the right ventricular free wall of the heart 610.

The can electrode 602 and the coil electrode 604 are positioned so that the majority of ventricular tissue is included within a volume defined between the can electrode 602 and the coil electrode 604. FIG. 3A illustrates a cross sectional area 605 formed by the lines drawn between active elements of the can electrode 602 and the coil electrode 604. Lines drawn between active areas of the electrodes 602, 604, may be defined by a medial edge and a lateral edge of the can electrode 602, and a proximal end and a distal end of a coil electrode utilized as the second electrode subsystem 604. The coil electrode 604 extends a predetermined distance beyond the apex of the heart 610, e.g. less than about 3 cm.

A similar configuration is illustrated in FIG. 3B. In this embodiment, the can electrode 602 is placed superior to the heart 610 in the right pectoral region. The coil electrode 604 is positioned inferior to the heart. In one arrangement, the coil electrode is positioned relative to an inferior aspect of the heart 610, for example, the apex of the heart. The can electrode 602 and the coil electrode 604 are positioned so that the majority of ventricular tissue is included within a volume defined between the can electrode 602 and the coil electrode 604.

FIG. 3B illustrates a cross sectional area 605 formed by the lines drawn between active elements of the can electrode 602 and the coil electrode 604. Lines drawn between active areas of the electrodes 602, 604, may be defined by a medial edge and a lateral edge of the can electrode 602, and a proximal end and a distal end of a coil electrode utilized as the second electrode subsystem 604. The coil electrode 604 extends a predetermined distance beyond the apex of the heart 610, e.g. less than about 3 cm.

FIG. 3C illustrates a configuration wherein the pulse generator housing 601 does not include an electrode. In this implementation two electrode subsystems are positioned about the heart so that a majority of ventricular tissue is included within a volume defined between the electrode subsystems. According to this embodiment, the first and second electrodes are configured as first and second coil electrodes 608, 609. The first coil electrode 608 is located superior to the heart 610 and may be positioned relative to a superior aspect of the heart, e.g., the left ventricular free wall. The second coil electrode 609 is located inferior to the heart 610. The second electrode 609 may be positioned in relation to an inferior aspect of the heart 610. In one configuration, the second electrode 609 is positioned parallel to the right ventricular free wall with a tip of the electrode 609 extending less than about 3 cm beyond the apex of the heart 610. As illustrated in FIG. 3C, the volume defined between the electrodes may be defined by the cross sectional area 605 bounded by lines drawn between active areas of the electrodes 608, 609.

Although one or both of the first and second electrode subsystems are illustrated in FIGS. 3A-C as coil electrode(s), the first and second electrode subsystems may additionally or alternatively comprise one or any combination of one or more coil electrodes, tip electrodes, ring electrodes, can electrodes, multiple coils, multi-element coils, spiral coils, spiral coils mounted on non-conductive backing, screen patch electrodes, and/or any other type of suitable electrode.

Any of the above electrode subsystems may include electrodes for pacing, sensing, and/or cardioversion/defibrillation. The can electrode may contain separate electrodes on its surface for pacing, sensing, and/or cardioversion/defibrillation. Alternatively, two or more of the pacing, sensing, and cardioversion/defibrillation electrodes may be integrated into a single electrode.

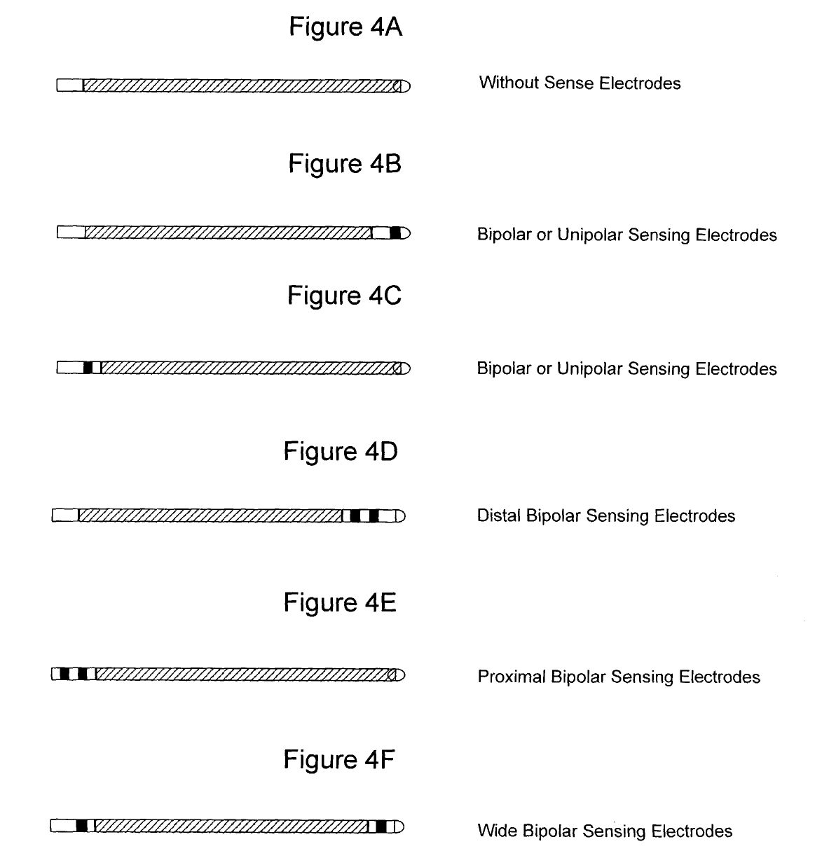

FIGS. 4A-F illustrate example configurations of electrode subsystems that may be used to sense the electrical activity of the heart and/or to provide electrical stimulation to the heart. In these example configurations, a lead system includes a coil electrode and may also include separate tip and ring electrodes distributed at various positions along its length, as indicated by FIGS. 4A-F.

One configuration (FIG. 4A) comprises a single coil without separate pace/sense electrodes. Another configuration (FIGS. 4B and 4C) illustrate a single ring and a single coil that may be used for integrated bipolar or unipolar sensing and stimulation. Other configurations include two rings and a coil electrode that may be positioned for sensing and pacing in a distal bipolar configuration (FIG. 4D), a proximal bipolar configuration (FIG. 4E) or a wide bipolar configuration (FIG. 4F). Other configurations of sensing, pacing and cardioversion/defibrillation electrodes including various combinations of tip, ring, coil, and other types of electrodes are also possible.

Various modifications and additions can be made to the preferred embodiments discussed hereinabove without departing from the scope of the present invention. Accordingly, the scope of the present invention should not be limited by the particular embodiments described above, but should be defined only by the claims set forth below and equivalents thereof.

What is claimed is:

1. A medical system, comprising:
a housing;
a medical device disposed within the housing; and
subcutaneous electrode subsystems coupled to the medical device, the electrode subsystems positioned relative to a heart so that a majority of ventricular tissue is included within a volume defined between the electrode subsystems.

2. The system of claim 1, wherein the volume defined between the electrode subsystems comprises a volume defined between active portions of the electrode subsystems.

3 The system of claim 1, wherein the volume defined between the electrode subsystems comprises a volume defined between a coil electrode and a can electrode.

4. The system of claim 1, wherein the volume defined between the electrode subsystems comprises a volume defined between a first coil electrode and a second coil electrode.

5. The system of claim 1, wherein the volume defined between the electrode subsystems comprises a volume defined between a first can electrode and a second can electrode.

6. The system of claim 1, wherein the volume defined between the electrode subsystems comprises a volume associated with a cross sectional area defined by ends of the electrode subsystems.

7. The system of claim 6, wherein the ends of the electrode subsystems include a medial edge and a lateral edge of a can electrode.

8. The system of claim 6, wherein the ends of the electrode subsystems include a proximal end and a distal end of a coil electrode.

9. The system of claim 1, wherein the housing is positioned subcutaneously.

10. The system of claim 1, wherein the housing is positioned in a left pectoral region.

11. The system of claim 1, wherein the housing is positioned in a right pectoral region.

12. The system of claim 1, wherein the housing is configured to have a volume ranging from about 20 cm³ to about 100 cm³.

13. The system of claim 1, wherein the housing is configured to have a surface area ranging from about 30 cm² to about 100 cm².

14. The system of claim 1, wherein the housing is configured to have a thickness ranging from about 0.4 cm to about 2 cm.

15. The system of claim 1, wherein the medical device comprises a diagnostic device.

16. The system of claim 1, wherein the medical device comprises a therapeutic device.

17. The system of claim 1, wherein the medical device comprises a monitoring device.

18. The system of claim 1, wherein the medical device comprises a cardiac rhythm management system.

19. The system of claim 1, wherein the medical device is configured to deliver pacing stimulation to the heart.

20. The system of claim 1, wherein the medical device is configured to deliver cardioversion/defibrillation stimulation to the heart.

21. The system of claim 1, wherein one or more of the electrode subsystems are configured to sense one or more physiological signals.

22. The system of claim 21, wherein the physiological signals are cardiac system signals.

23. The system of claim 21, wherein the physiological signals are respiratory system signals.

24. The system of claim 21, wherein the physiological signals are patient activity signals.

25. The system of claim 1, wherein one or more of the electrode subsystems comprise at least one coil electrode.

26. The system of claim 1, wherein one or more of the electrode subsystems comprise at least one electrode having multiple coils.

27. The system of claim 1, wherein one or more of the electrode subsystems comprise at least one spiral coil electrode.

28. The system of claim 1, wherein one or more of the electrode subsystems comprise at least one coil electrode mounted on a non-conductive substrate.

29. The system of claim 1, wherein one or more of the electrode subsystems comprise at least one screen patch electrode.

30. The system of claim 1, wherein one or more of the electrode subsystems comprise a coil electrode having a length ranging from about 5 cm to about 12 cm.

31. The system of claim 1, wherein one or more of the electrode subsystems comprise a coil electrode having a preformed curve.

32. The system of claim 1, wherein one or more of the electrode subsystems comprise a coil electrode having a diameter ranging from about 3 French to about 15 French.

33. The system of claim 1, wherein at least one of the electrode subsystems is coupled to the medical device through a lead.

34. The system of claim 1, wherein at least one of the electrode subsystems comprises a first electrode located at a distal end of a lead and a second electrode located proximate the first electrode.

35. The system of claim 34, wherein the second electrode comprises a coil electrode.

36. The system of claim 34, wherein the first electrode comprises a ring electrode and the second electrode comprises a coil electrode.

37. The system of claim 34, wherein the first electrode comprises a coil electrode and the second electrode comprises a ring electrode.

38. A medical system, comprising:
a medical device disposed within a housing, the housing comprising a can electrode; and
a subcutaneous electrode subsystem coupled to the medical device, wherein the can electrode and the electrode subsystem are positioned relative to a heart so that a majority of ventricular tissue is included within a volume defined between the can electrode and the electrode subsystem.

39. The system of claim 38, wherein the housing is positioned subcutaneously.

40. The system of claim 38, wherein the housing is positioned in a right pectoral region and the electrode subsystem is positioned in relation to an inferior aspect of the heart.

41. The system of claim 38, wherein the housing is positioned in a right pectoral region and the electrode subsystem is positioned in relation to an apex of the heart.

42. The system of claim 38, wherein the housing is positioned in a left pectoral region and the electrode subsystem is positioned in relation to an inferior aspect of the heart.

43. The system of claim 38, wherein the housing is positioned in a left pectoral region and the electrode subsystem is positioned substantially parallel to a right ventricular free wall.

44. The system of claim 43, wherein one end of the electrode subsystem extends a predetermined distance beyond an apex of the heart.

45. The system of claim 44, wherein the predetermined distance is less than about 3 cm.

46. The system of claim 38, wherein the electrode subsystem comprises a coil electrode.

47. The system of claim 46, wherein the length of the coil electrode ranges from about 5 cm to about 12 cm.

48. The system of claim 38, wherein the majority of ventricular tissue is included within a volume defined by medial and lateral edges of the can electrode and proximal and distal ends of the coil.

49. A medical system, comprising:
a housing;
a medical device disposed within the housing; and
first and second subcutaneous electrode subsystems coupled to the medical device, wherein the first and the second subcutaneous electrode subsystems are positioned relative to a heart so that a majority of ventricular tissue is included within a volume defined between the first and the second electrode subsystems.

50. The system of claim 49, wherein the first electrode subsystem is positioned in relation to a superior aspect of the heart and the second electrode subsystem is positioned in relation to an inferior aspect of the heart.

51. The system of claim 49, wherein the first electrode subsystem is positioned in relation to a left ventricle and the second electrode subsystem is positioned in relation to a right ventricle.

52. The system of claim 49, wherein the first electrode subsystem is positioned substantially parallel to a left ventricular free wall.

53. The system of claim 52, wherein one end of the first electrode subsystem extends a predetermined distance beyond an apex of the heart.

54. The system of claim 53, wherein the predetermined distance is less than about 3 cm.

55. The system of claim 49, wherein the second electrode subsystem is positioned substantially parallel to a right ventricular free wall.

56. The system of claim 49, wherein one end of the second electrode subsystem extends a predetermined distance beyond an apex of the heart.

57. The system of claim 56, wherein the predetermined distance is less than about 3 cm.

58. The system of claim 49, wherein the first electrode subsystem comprises a first coil electrode and the second electrode subsystem comprises a second coil electrode.

59. The system of claim 58, wherein the majority of ventricular tissue is included within a volume defined between respective proximal and distal ends of the first coil electrode and respective proximal and distal ends of the second coil electrode.

60. An electrode system, comprising:
a first subcutaneous electrode subsystem; and
a second subcutaneous electrode subsystem positionable so that a majority of ventricular tissue is included within a volume defined between the first and the second electrode subsystems.

61. The system of claim 60, wherein the first electrode subsystem is positionable in relation to a superior aspect of the heart and the second electrode subsystem is positionable in relation to an inferior aspect of the heart.

62. The system of claim 60, wherein the first electrode subsystem is positionable in relation to a left ventricle and the second electrode subsystem is positionable in relation to a right ventricle.

63. The system of claim 60, wherein the first electrode subsystem is positionable substantially parallel to a left ventricular free wall.

64. The system of claim 60, wherein one end of the first electrode subsystem extends a predetermined distance beyond an apex of the heart.

65. The system of claim 60, wherein at least one of the electrode subsystems comprises a can electrode.

66. The system of claim 60, wherein at least one of the electrode subsystems comprises a coil electrode.

67. The system of claim 60, wherein at least one of the electrode subsystems comprises a spiral coil.

68. The system of claim 60, wherein at least one of the electrode subsystems comprises a spiral coil mounted on a non-conductive substrate.

69. The system of claim 60, wherein at least one of the electrode subsystems comprises a screen patch electrode.

70. The system of claim 60, wherein at least one of the electrode subsystems comprises a coil electrode having a length ranging between about 5 cm and about 12 cm.

71. The system of claim 60, wherein at least one of the electrode subsystems comprises a coil electrode having a preformed curve.

72. The system of claim 60, wherein at least one of the electrode subsystems comprises a coil electrode having a diameter ranging between about 3 French and about 15 French.

73. The system of claim 60, wherein at least one of the electrode subsystems comprises a first electrode located at a distal end of a lead.

74. The system of claim 73, wherein the at least one electrode subsystem further comprises a second electrode located proximate the first electrode.

75. The system of claim 74, wherein the second electrode comprises a coil electrode.

76. The system of claim 74, wherein the first electrode comprises a ring electrode and the second electrode comprises a coil electrode.

77. The system of claim 74, wherein the first electrode comprises a coil electrode and the second electrode comprises a ring electrode.

78. The system of claim 60, wherein at least one of the electrode subsystems comprises a first electrode located at a distal end of a lead, a second electrode located proximate the first electrode, and a third electrode located proximate the second electrode.

79. The system of claim 78, wherein the first electrode comprises a coil electrode.

80. The system of claim 78, wherein the first electrode comprises a coil electrode and the second and third electrodes comprise ring electrodes.

81. The system of claim 78, wherein the third electrode comprises a coil electrode.

82. The system of claim 78, wherein the third electrode comprises a coil electrode and the first and second electrodes comprise ring electrodes.

83. The system of claim 78, wherein the second electrode comprise a coil electrode.

84. The system of claim 78, wherein the second electrode comprises a coil electrode and the first and third electrodes comprise ring electrodes.

85. A method, comprising:
providing subcutaneous electrode subsystems coupled to a medical device disposed within a housing; and
positioning the electrode subsystems in relation to a heart so that a majority of ventricular tissue is included within a volume defined between the electrode subsystems.

86. The method of claim 85, further comprising sensing physiological signals using the electrode subsystems.

87. The method of claim 86, wherein sensing the physiological signals comprises sensing respiratory system signals.

88. The method of claim 86, wherein sensing the physiological signals comprises sensing cardiac system signals.

89. The method of claim 86, wherein sensing the physiological signals comprises sensing signals associated with patient activity.

90. The method of claim 86, wherein sensing the physiological signals comprises sensing transthoracic impedance signals.

91. The method of claim 86, further comprising monitoring one or more patient conditions using the sensed physiological signals.

92. The method of claim 85, further comprising sensing electrical activity of the heart using the subcutaneous electrode subsystems.

93. The method of claim 85, further comprising delivering electrical stimulation to the heart using the subcutaneous electrode subsystems.

94. The method of claim 93, wherein delivering electrical stimulation to the heart comprises delivering cardioversion/defibrillation stimulation.

95. The method of claim 93, wherein delivering electrical stimulation to the heart comprises delivering pacing stimulation.

96. The method of claim 85, wherein positioning the electrode subsystems comprises:
positioning a first electrode subsystem in relation to a superior aspect of the heart; and
positioning a second electrode subsystem in relation to an inferior aspect of the heart.

97. The method of claim 96, wherein positioning the first electrode subsystem comprises positioning the first electrode subsystem on the housing and positioning the housing subcutaneously in a pectoral region.

98. The method of claim 85, wherein positioning the electrode subsystems comprises positioning at least one electrode subsystem substantially parallel to a ventricular free wall.

99. The method of claim 85, wherein positioning the electrode subsystems comprises positioning at least one electrode subsystem parallel to a ventricular free wall and extending a predetermined distance beyond the apex of the heart.

100. The method of claim 99, wherein the predetermined distance is less than about 3 cm.

101. A medical device, comprising:
means for sensing physiological conditions;
means for detecting cardiac arrhythmia based on the sensed physiological conditions; and
means for electrically stimulating a heart to mitigate the cardiac arrhythmia, the means for electrically stimulating the heart positioned subcutaneously in relation to the heart so that a majority of ventricular tissue is included within a volume defined between the means for electrically stimulating the heart.
